(19) United States
(12) Patent Application Publication
(10) Pub. No.: US 2024/0027336 A1
(43) Pub. Date: Jan. 25, 2024

(54) NITRIC OXIDE MEASUREMENT

(71) Applicant: NINOX MEDICAL LTD., Ein Ayala (IL)

(72) Inventors: Dmitry MEDVEDEV, Moscow (RU); Roman ILIEV, Moscow (RU); Boris MISLAVSKY, Moscow (RU)

(21) Appl. No.: 18/266,059

(22) PCT Filed: Dec. 9, 2021
(86) PCT No.: PCT/IL21/51478

§ 371 Date: Jun. 8, 2023

Related U.S. Application Data
(60) Provisional application No. 63/123,166, filed on Dec. 9, 2020.

(51) Int. Cl. G01N 21/33; G01N 33/00

(52) U.S. Cl. CPC G01N 21/33; G01N 33/0027; G01N 2201/062; G01N 2201/0636

(57) ABSTRACT

A sensor for measuring nitric oxide concentration in a sample, the sensor constituted of: an ozone source for oxidizing nitric oxide within a sample to form NO₂; and one or more light adsorption measurement systems for determining NO₂ levels in the sample in the nitric oxide analyzer before and after oxidizing.

### CROSS REFERENCE TO RELATED APPLICATIONS

The present application claims priority from U.S. patent application Ser. 63/123,166, filed Dec. 9, 2020, and entitled “Nitric Oxide Measurement”, the entire contents of which incorporated herein by reference.

### TECHNICAL FIELD

The present disclosure relates to the measurement of nitric oxide gas, particularly as related to nitric oxide therapies.

### BACKGROUND

Nitric oxide therapy has shown promise in several areas of medicine, especially in the field of pulmonology. In particular, research has shown that nitric oxide therapy may aid in the treatment of pulmonary arterial hypertension (PAH). PAH is a sometimes-fatal condition characterized by increased blood pressure in the lungs resulting from obstructions in the arteries of the lung. Pharmacological treatment of PAH is not particularly effective, with at least 50% of patients dying during 2-5 years depending on the stage of the disease. While the precise mechanisms of disease progression are not entirely clear, several factors have been implicated in the pathology of PAH. One of the most important mediators is Nitric Oxide (NO), a lack of which has been found to contribute to pulmonary artery vasoconstriction, vascular remodeling, and right ventricular failure associated with PAH pathology.

The vasodilator and anti-proliferative actions of NO make it an attractive tool for pharmacological treatment of PAH. Administration of NO gas by inhalation has been shown to be beneficial to patients with PAH, particularly in children with congenital heart diseases. However, inhaled NO therapies are hampered by high costs, technical difficulties, and inconsistent patient response. Rapid withdrawal of inhaled NO therapy can also have deleterious effects with levels of oxygenation and pulmonary hypertension returning to levels worse than those seen prior to the commencement of therapy.

Nitric oxide has other possible applications in gene therapy. Currently, gene-based therapy is recognized as a powerful new therapeutic weapon for treating pulmonary arterial hypertension. Genetic manipulation may be supplemental to standard pharmacotherapy or be used as a stand-alone treatment. However, genetic material must be transferred into cells and expressed at a desired level to provide therapeutic benefits. NO may play a role in improving gene transduction in gene therapies for treating PAH.

Accurate NO level sensing is paramount for NO generation, especially for medical applications. Numerous approaches have been used and proposed for monitoring the concentration of nitric oxide in a gas mixture. Existing methods include mass spectroscopy, electrochemical analysis, calorimetric analysis, chemiluminescence analysis, and piezoelectric resonance techniques. However, each of these approaches have shortcomings that make them poorly suited for widespread use in the diagnosis and treatment of disease.

Mass spectroscopy utilizes a mass spectrometer to identify particles present in a substance. The particles are ionized and beamed through an electromagnetic field. The manner in which the particles are deflected is indicative of their mass, and thus their identity. Mass spectroscopy is accurate but requires the use of very expensive and complicated equipment. Also, the analysis is relatively slow, making it unsuitable for real time analysis of produced or delivered NO levels. Preferably, in the breath-by-breath analysis of nitric oxide, it is desirable to quickly and accurately measure the nitric oxide concentration in the flow path as the gas mixture flows through the flow path. However, mass spectroscopy requires sampling of portions of the gas mixture rather than analyzing the nitric oxide concentration in the flow pathway itself. Mass spectroscopy cannot be considered an instantaneous or continuous analysis approach. Such sampling-based systems are especially deficient when detecting gases in very low concentrations since large samples are required.

Electrochemical-based analysis systems use an electrochemical gaseous sensor in which gas from a sample diffuses into and through a semi-permeable barrier, such as a membrane, then through an electrolyte solution, and then to one of typically three electrodes. At one of the three electrodes, a sensing redox reaction occurs. At the second, counter, electrode, a complimentary and opposite redox reaction occurs. A third electrode is typically provided as a reference electrode. Upon oxidation, or reduction, of the nitric oxide at the sensing electrode, a current flows between the sensing and counter electrode that is proportional to the amount of nitric oxide reacting at the sensing electrode surface. The reference electrode is used to maintain the sensing electrode at a fixed voltage. A typical electrochemical-based gas analyzer for detecting nitric oxide is shown in U.S. Pat. No. 5,565,075 to Davis et al, incorporated herein by reference. Electrochemical-based devices have high sensitivity and accuracy but require frequent calibration and associated service costs and delays.

Chemiluminescent-based sensors depend on the oxidation of nitric oxide by mixing nitric oxide with ozone, O₃, to create nitrogen dioxide (NO₂) and oxygen. The nitrogen dioxide is in an excited state immediately following the reaction and releases photons as it decays back to a non-excited state. By sensing the amount of light emitted during this reaction, the concentration of nitric oxide may be determined. An example of a chemiluminescent-based device is shown in U.S. Pat. No. 6,099,480 to Gustafsson, incorporated herein by reference. Chemiluminescent devices are typically very large and expensive and their accuracy is sensitive to environmental factors.

The most convenient and reliable gas analysis method for sensors of this field is direct optical measurements of gas components by adsorption of light at certain wave lengths. The main advantage of this method is stability of adsorption in time because the adsorption coefficient is fundamentally constant. Accordingly, stable measurements can be provided without frequent calibration so long as the optical instruments are kept clean. Current gas analyzers 10 based on light adsorption (see FIG. 1) consist of: a light source 20 which generates radiation on a wavelength which is adsorbed by the gas component to be measured; an optical cuvette 25 to allow light to pass through the contained gas, the optical cuvette comprising a sealing 26 and optical window 27 on each end; a gas input 30; a gas output 40; and a light sensor 50, fed by a lens 55, which can transform the light passed through the gas from the light source 20 into a voltage signal. Suitable light sources 20 include LEDs and laser diodes and suitable light sensors 50 include photo diodes, photo resistors, or phototransistors which have practically unlimited service lifetimes and sufficiently stable characteristics. The wavelength of the emitted light can be selected as one that is adsorbed by the target gas component and the light sensor 50 can measure the light intensity after the emitted light has passed through the gas. Thus, the adsorption and associated gas component concentration can be determined. Unfortunately, nitric oxide does not have adsorption bands in the visible light and near UV spectrums, rendering this method inapplicable to nitric oxide measurement.

### SUMMARY

Accordingly, it is a principal object of the present invention to overcome at least some of the disadvantages of prior art plasma generation systems. This is provided in one embodiment by a sensor for measuring nitric oxide concentration in a sample, the sensor comprising: an ozone source for oxidizing nitric oxide within a sample to form NO₂; and one or more light adsorption measurement systems for determining NO₂ levels in the sample in the nitric oxide analyzer before and after oxidizing.

In one embodiment, the light adsorption measurement system comprises a light source positioned to pass light through the sample within the sensor. In another embodiment, the sensor further comprises a light sensor positioned to receive light from the light source passed through the sample within the sensor.

In one embodiment, the light source emits light having a wavelength of about 350 nm to about 400 nm. In another embodiment, the light source comprises one or more LEDs.

In one embodiment, the sensor further comprises a processor configured to receive adsorption data from the one or more light adsorption measurement systems and determine an NO₂ level therefrom. In another embodiment, the sensor comprises one or more mirrors for reflecting light to pass through the sample one or more times before entering the light sensor, thereby increasing the beam length for measurement of low concentrations of NO₂.

In one embodiment, a first light adsorption measurement system is positioned upstream of the ozone source and a second light adsorption measurement system is positioned downstream of the ozone source.

In another embodiment, the processor is in communication with the ozone source and is configured to control ozone introduction to the sample through a valve or pump and to determine NO₂ levels before and after introducing ozone to the sample.

In one independent embodiment, a method for measuring nitric oxide concentration in a sample is provided, the method comprising: oxidizing nitric oxide within a volume of sample using ozone to form NO₂; measuring light adsorption by NO₂ within the sample to determine NO₂ levels in the sample in the nitric oxide analyzer before and after oxidizing; and subtracting NO₂ levels determined before oxidizing from NO₂ levels determined after oxidizing to determine a nitric oxide concentration in the sample.

In one embodiment, the method further comprises passing light through the sample from a light source within the sensor. In another embodiment, the method further comprises measuring light intensity in light from the light source passed through the sample within the sensor using a light sensor.

In one embodiment, the light source emits light having a wavelength of about 350 nm to about 400 nm. In another embodiment, the light source comprises one or more LEDs.

In one embodiment, a first light adsorption measurement system is positioned upstream of the ozone source and a second light adsorption measurement system is positioned downstream of the ozone source, the method comprising subtracting NO₂ levels from the first light adsorption measurement system from NO₂ levels from the second light adsorption measurement system.

In another embodiment, the method further comprises measuring NO₂ levels in the sample, then introducing ozone the sample, then measuring NO₂ levels in the sample again to determine NO₂ levels before and after oxidizing.

In one embodiment, the method further comprises passing the light through the sample a plurality of times before receiving the light with the light sensor.

In one embodiment, determining nitric oxide levels is according to the formula C2*(C2N/C1N)−C1, where: C1N is the NO₂ level from the first light adsorption measurement system before introduction of ozone; C2N is the NO₂ level from the second light adsorption measurement system before introduction of ozone, C1 is the NO₂ level from the first light adsorption measurement system after oxidation with ozone; and C2 is the NO₂ level from the second light adsorption measurement system after oxidation with ozone.

In another embodiment, the method further comprises cyclically introducing ozone to the sample to oxidize the NO₂ therein, wherein determining nitric oxide levels is according to the formula C_{NO}=(Ln(Iₘₐₓ/Iₘᵢₙ))*Kcal−C_{NO2}, where: C_{NO2}=(Ln(Iₘₐₓ/Iᵢₙ))*Kcal; Iᵢₙ is initial light intensity of light passed through the sample at the beginning of an ozone introduction cycle; Iₘᵢₙ is minimal light intensity of light passed through the sample during the ozone introduction cycle; Iₘₐₓ is maximal light intensity of light passed through the sample during the ozone introduction cycle; and Kcal is a calibrating coefficient.

Additional features and advantages of the invention will become apparent from the following drawings and description.

Unless otherwise defined, all technical and scientific terms used herein have the same meaning as commonly understood by one of ordinary skill in the art to which this invention pertains. In case of conflict, the patent specification, including definitions, governs. As used herein, the articles “a” and “an” mean “at least one” or “one or more” unless the context clearly dictates otherwise. As utilized herein, “and/or” means any one or more of the items in the list joined by “and/or”. As an example, “x and/or y” means any element of the three-element set {(x), (y), (x, y)}. In other words, “x and/or y” means “x, y or both of x and y”. As another example, “x, y, and/or z” means any element of the seven-element set {(x), (y), (z), (x, y), (x, z), (y, z), (x, y, z)}.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of embodiments of the instant inventive concepts. This is done merely for convenience and to give a general sense of the inventive concepts, and “a” and “an” are intended to include one or at least one and the singular also includes the plural unless it is obvious that it is meant otherwise.

As used herein, the term “about”, when referring to a measurable value such as an amount, a temporal duration, and the like, is meant to encompass variations of +/−10%, more preferably +/−5%, even more preferably +/−1%, and still more preferably +/MI % from the specified value, as such variations are appropriate to perform the disclosed devices and/or methods.

The following embodiments and aspects thereof are described and illustrated in conjunction with systems, tools and methods which are meant to be exemplary and illustrative, but not limiting in scope. In various embodiments, one or more of the above-described problems have been reduced or eliminated, while other embodiments are directed to other advantages or improvements.

### BRIEF DESCRIPTION OF THE DRAWINGS

For a better understanding of the invention and to show how the same may be carried into effect, reference will now be made, purely by way of example, to the accompanying drawings in which like numerals designate corresponding sections or elements throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice. In the accompanying drawings:

FIG. 1 shows a light-adsorption-based concentration sensor, in accordance with the prior art;

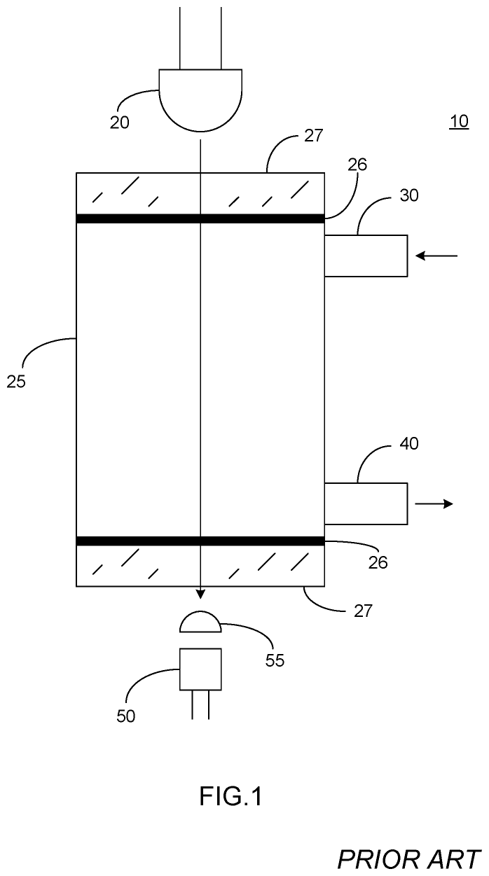

FIG. 2 shows an exemplary NO sensor including two NO₂ sensors and ozone generator, in accordance with some embodiments of the disclosure;

FIG. 3 illustrates exemplary ozone capacity modulation, in accordance with some embodiments of the disclosure;

FIG. 4 shows an exemplary NO sensor including a single NO₂ sensor and ozone generator, in accordance with some embodiments of the disclosure;

FIG. 5 shows exemplary ozone capacity modulation for a sensor such as that depicted in FIG. 4, in accordance with some embodiments of the disclosure;

FIG. 6 shows an exemplary NO sensor for measuring concentrations in two independent gas flows, in accordance with some embodiments of the disclosure; and

FIGS. 7A-7B show an exemplary sensor comprising parallel mirrors for increased optical beam length, in accordance with some embodiments of the disclosure.

### DETAILED DESCRIPTION OF CERTAIN EMBODIMENTS

Before explaining at least one embodiment in detail, it is to be understood that the invention is not limited in its application to the details of construction and the arrangement of the components set forth in the following description or illustrated in the drawings. The invention is applicable to other embodiments being practiced or carried out in various ways. Also, it is to be understood that the phraseology and terminology employed herein is for the purpose of description and should not be regarded as limiting.

Systems and methods of the disclosure provide more accurate and responsive nitric oxide sensors useful in providing fast feedback for control of nitric oxide generation in the medical field as well as other areas. As discussed above, light adsorption systems are the most desirable from a standpoint of ease-of-use, affordability, accuracy, and packaging but are not readily applied to nitric oxide. NO₂, however, is readily detectable using such methods, having adsorption bands in the 400 nm wavelength range. Accordingly, in certain embodiments systems and methods of the disclosure may oxidize NO to NO₂ and then use light adsorption sensors to measure the level of NO₂ which can then be used to infer the amount of NO in the system. Multiple sensors may be used to determine pre and post oxidation levels of NO₂ in the sample gas to provide a more accurate analysis of what amount of post-oxidized NO₂ level is attributable to oxidized NO.

Systems and methods may include nitric oxide analyzers positioned in a measurement line comprising a pump and an outlet to vent measured gas from the system. Such analyzers may include an ozone source for oxidizing nitric oxide to form NO₂ within the nitric oxide analyzer and one or more light adsorption measurement systems for determining NO₂ levels in gas in the nitric oxide analyzer before and after oxidizing. The light adsorption measurement system may include a light source positioned to pass light through the sample and a light sensor positioned to receive light passed therethrough. The light may have a wavelength in the range of about 350 nm to about 400 nm and may come from, for example, an LED. The sensor may comprise a transparent portion to allow light to enter and leave the interior of the sample-filed sensor.

A computer system may be in communication with the light adsorption measurement system in order to receive adsorption data therefrom and calculate NO levels accordingly. In certain embodiments, a plurality of light adsorption measurement systems may be used with positioning before and after the ozone source in order to establish a baseline level of NO₂.

Sensors of the disclosure may include one or more mirrors for reflecting light to pass through the sample one or more times before entering the light sensor, thereby increasing the beam length for measurement of low concentrations of NO₂. Accordingly, small concentrations of NO₂ can be detected in narrow sensor chambers.

In various embodiments, nitric oxide levels may be determined using the equation:

C2*(C2N/C1N)−C1 EQ. 1

where: C1N is the NO₂ level from the first light adsorption measurement system before introduction of ozone; C2N is the NO₂ level from the second light adsorption measurement system before introduction of ozone; C1 is the NO₂ level from the first light adsorption measurement system after oxidation with ozone; and C2 is the NO₂ level from the second light adsorption measurement system after oxidation with ozone.

In certain embodiments, nitric oxide levels may be determined according to equation:

C_{NO}=(Ln(Iₘₐₓ/Iₘᵢₙ))*Kcal−C_{NO2} EQ. 2

where: C_{NO2}=(Ln(Iₘₐₓ/Iᵢₙ))*Kcal; Iᵢₙ is initial light intensity of light passed through the sample at the beginning of an ozone introduction cycle; Iₘᵢₙ is the minimal light intensity of light passed through the sample during the ozone introduction cycle; Iₘₐₓ is the maximal light intensity of light passed through the sample during the ozone introduction cycle; and Kcal is a calibrating coefficient.

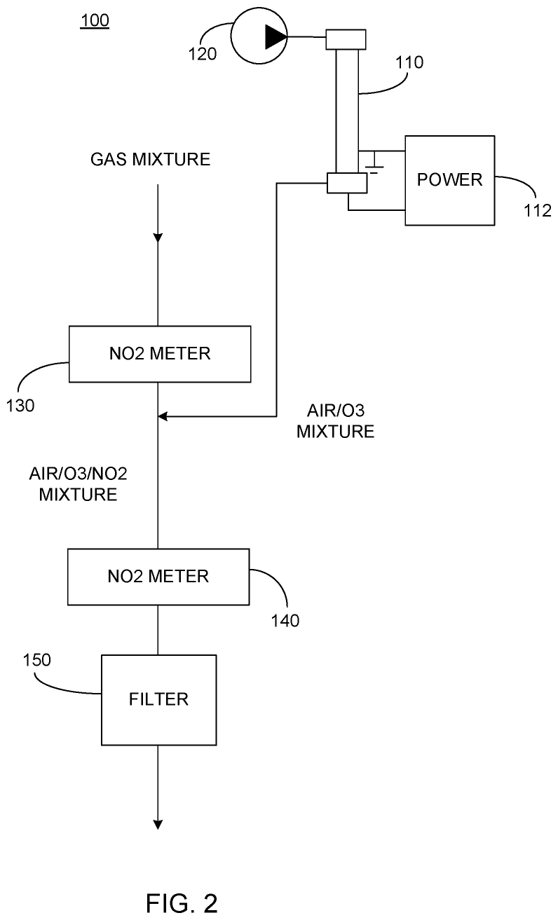

As described above, accurate measurement of NO levels is important in many applications and especially in the medical field where inaccurate measurements can have serious implications to patient health. Systems and methods of the disclosure provide accurate and fast acting NO sensors for determining NO concentration in output gas from NO generators as well as other sources including in patient exhalation. In preferred embodiments, such sensors rely on the oxidation of NO to NO₂ by, for example, the introduction of ozone to the output gas, as shown in FIG. 2.

Particularly, FIG. 2 shows a sensor 100 comprising: an ozone generator 110 configured to generate ozone, optionally comprising a power supply 112; an air pump 120; an NO₂ meter 130 configured to measure the amount of NO₂ flowing therethrough; an NO₂ meter 140 configured to measure the amount of NO₂ flowing therethrough; and an optional NO₂ and/or NO filter 150. An input of ozone generator 110 is coupled to an output of air pump 120. An output of ozone generator 110 is in fluid communication with an output of NO₂ meter 130 and an input of NO₂ meter 140. The term “fluid communication”, as used herein, means that a path exists between the two components such that fluid can flow therebetween. Fluid can include, liquid, gas and/or plasma. A gas mixture containing NO is input into NO₂ meter 130 to measure the initial amount of NO₂ in the mixture. A mixture of air and O₃ is then added to the mixture and then measured again by NO₂ meter 140. As described above, the O₃ converts NO into NO₂. Therefore, the difference between the amount of NO₂ measured by NO₂ meter 140 and the amount of NO₂ measured by NO₂ meter 130 is indicative of the amount of NO in the initial mixture.

To calculate NO concentration by proxy of NO₂ concentration after oxidation of NO by ozone, a baseline NO₂ concentration may be established. To do this NO₂ concentration may be measured optically in a first cuvette (e.g. NO₂ meter 130) before oxidation and then after ozone flow admixing in a second cuvette (e.g. NO₂ meter 140).

For optical measurements of NO₂ concentration, light radiation emitted by, for example, an LED with a wavelength of about 400 nm is passed through the optical cuvette. NO₂ concentration can be calculated based on observed light adsorption as follows:

I=Io*exp(−K*Cno2) EQ. 3

where I is light intensity after absorption, Io is light intensity without absorption (with zero NO₂ concentration), Cno2 is NO₂ concentration and K is a predetermined coefficient depending on the wavelength of light and units used and is proportional to cuvette length.

NO₂ concentration may be calculated by the following procedure. First, the device may be zeroed by taking a base reading. In zeroing, the NO₂ concentration in the cuvette is in one embodiment zero. For zeroing, the controller can take digital readings (Uo) of the signal from the amplifier amplifying the signal from the light sensor while NO₂ concentration in the cuvette is zero. Using Uo, the following calculation is made:

N=Ln(Umax/Uo) EQ. 4

where Umax is maximal and Uo is the digital readings from zeroing.

Then the NO₂ concentration can be calculated by:

C=(Ln(Umax/Uav)−N)*Kcal EQ. 5

where Uav is the average of actual digital readings of an ADC obtained during a certain time (time averaging can be entered in a program menu), Kcal is a calibrating coefficient (can be adjusted during calibration).

If the NO₂ concentration is still zero and Uav is equal to Uo, NO₂ readings are zero. In other cases the readings will be proportional to the NO₂ concentration in the cuvette and can be made equal to the actual NO₂ concentration by changing Kcal. NO concentration is calculated by comparison of readings in the first and second cuvettes by the following steps.

Two zeroing processes may be completed. In one embodiment, both cuvette channels are zeroed as described above. At initialization, the NO₂ concentration in both cuvettes are in one embodiment zero. A mixture of NO and NO₂ is then injected into the system. Ozone capacity is still set to zero. Then the NO₂ concentration in both cuvettes is measured, as described above, and saved to memory as C1N and C2N. Then zeroing can be finished and the operation mode can start. In the operation mode, the sensor can calculate NO concentration by the following formula:

NO=C2*(C2N/C1N)−C1 EQ. 6

where C2 and C1 are the current NO₂ readings from the first and second cuvette channels. The algorithm may be corrected to remove the influence of any oxidation of NO₂ by ozone. Fortunately, the reaction rate of ozone with NO is faster than the reaction of ozone with NO₂. The rate constant of NO+O₃→NO₂+O₂ in the temperature range of 283-443 K was published in August 1980 by H. H. Lippmann, et al. The reaction NO+O₃→NO₂+O₂ has been studied in a 220-m³ spherical stainless-steel reactor under stopped-flow conditions below 0.1 mtorr total pressure. Under the conditions used, the mixing time of the reactants was negligible compared with the chemical reaction time. The pseudo-first-order decay of the chemiluminescence owing to the reaction of ozone with a large excess of nitric oxide was measured with an infrared sensitive photomultiplier. One hundred twenty-nine decays at 18 different temperatures in the range of 283-443 K were evaluated. A weighted least-squares fit to the Arrhenius equation yielded k=(4.3±0.6)×10⁻¹² exp[−(1598±50)/T] cm³/molecule sec (two standard deviations in brackets). The Arrhenius plot showed no curvature within experimental accuracy. Comparison with recent results suggests that a nonlinear fit, as proposed by these authors, is more appropriate over an extended temperature range.

The rate constant for the reaction O₃+NO₂→O₂+NO₃ over the temperature range 259-362° K was published by Robert E. Huie, et al, in an article titled “The rate constant for the reaction O₃+NO₂→O2+NO3 over the temperature range 259-362° K”, published August 1974. The rate constant for the reaction of ozone with nitrogen dioxide has been measured over the temperature range 259 to 362° K, using a stopped-flow system coupled to a beam sampling mass spectrometer. A fit of the data to the Arrhenius equation gave:

k=(9.44±2.46)×10¹⁰ exp[(−2509±76)/T]cm3mol−1 sec−1 EQ. 7

So, the reaction rate of NO+O3 is: k=(4.3±0.6)×10⁻¹² exp[−(1598±50)/T] cm³/molecule sec=2.15 10⁻¹⁴ cm³/molecule sec for T=300 K, and the reaction rate of NO2+O3 is: k=(9.44±2.46)×10¹⁰ exp[(−2509±76)/T] cm3 mol−1 sec−1=0.157×10⁻¹² exp[(−2509±76)/T]=0.0036×10⁻¹⁴ cm³/molecule sec.

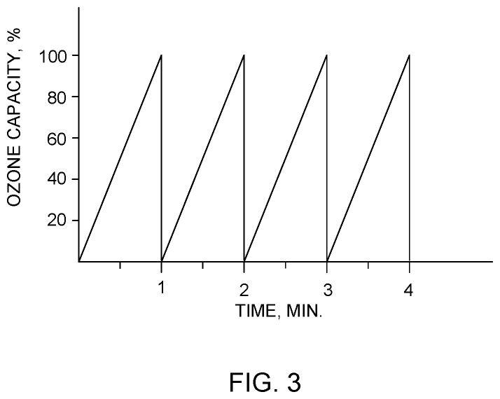

Accordingly, the reaction rate of NO with ozone is about 500 times faster than the reaction rate of NO₂ with ozone and NO₂ will start to react only when NO is completely oxidized. To find this moment, ozone capacity can be modulated as shown in the graph of FIG. 3.

During an ozone modulation cycle (e.g., over one minute), NO concentration can be calculated by the formula above. Ozone levels can initially increase and then start to decrease after the moment of complete oxidation of NO and beginning of oxidation of NO₂. The maximal concentration of NO calculated in the cycle is accepted as the level of NO concentration.

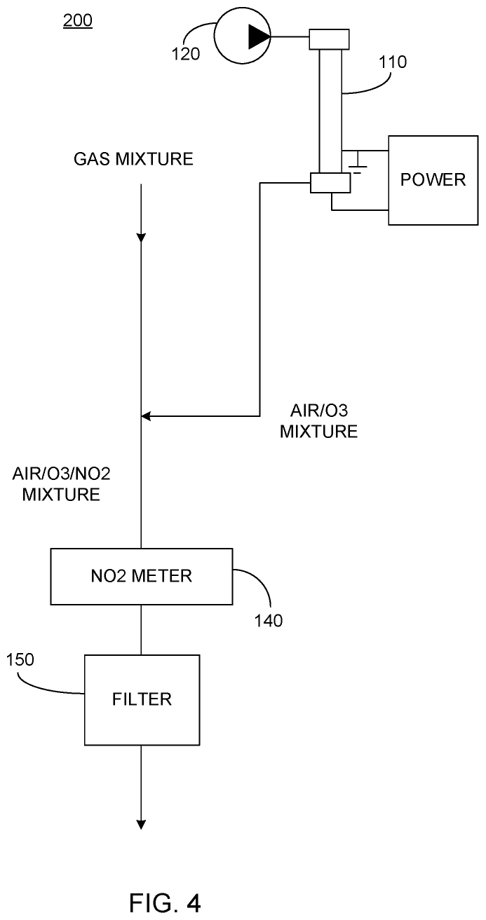

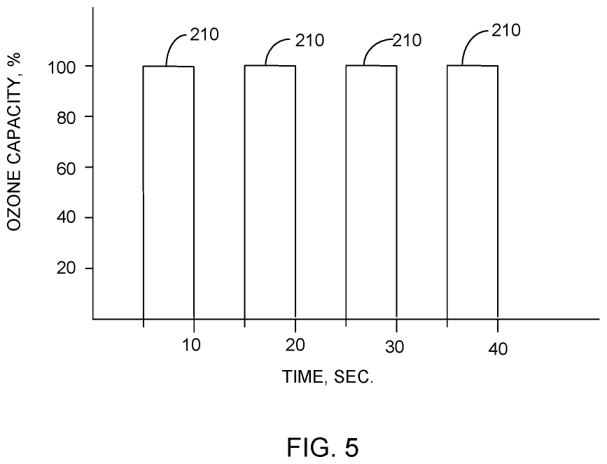

In a second embodiment, as shown in FIG. 4, only one optical cuvette is used. FIG. 4 shows a sensor 200, which is in all respects similar to sensor 100, with the exception that NO₂ meter 130 is not provided and NO₂ is measured only by NO₂ meter 140. Ozone is admixed before the gas enters the cuvette and mixed with analyzed gas flow. The ozone generator capacity is modulated in this embodiment in a different manner than the previous example. Instead of linearly increasing the ozone capacity, the ozone generator 110 is turned on and off periodically as shown by the pulses 210 in FIG. 5. In this case, ozone concentration increase is determined based on the time after the ozone generator 110 is turned on. In this case, measurement time can be up to ten times less than in the first embodiment. The measurement algorithm in this case is also different. At the moment the ozone generator 110 is turned on, the control unit records 400 nm light intensity as passed through the cuvette of meter 140 (Iᵢₙ). That intensity corresponds to adsorption by the initial NO₂ in the gas flow. The control unit can then detect the minimal intensity during operation of the ozone generation cycle (Iₘᵢₙ). This intensity corresponds to adsorption of total NO₂ including that initially present in the sample and that generated by NO oxidation. After minimum intensity starts to rise because of NO₂ oxidation, the control unit can detect maximal intensity ozone generator operation cycle Iₘₐₓ. Zero adsorption intensity corresponds to the moment when NO₂ concentration is zero. NO and NO₂ concentrations C_{NO} and C_{NO2} can be calculated as:

C_{NO2}=(Ln(Iₘₐₓ/Iᵢₙ))*Kcal C_{NO}=Ln(Iₘₐₓ/Iₘᵢₙ))*Kcal−C_{NO2} EQ. 8

where Iᵢₙ is initial light intensity during ozone generator operation cycle, Iₘᵢₙ is minimal light intensity during ozone generator operation cycle, Iₘₐₓ is maximal light intensity during ozone generator operation cycle and Kcal is a calibrating coefficient (can be adjusted during device calibration).

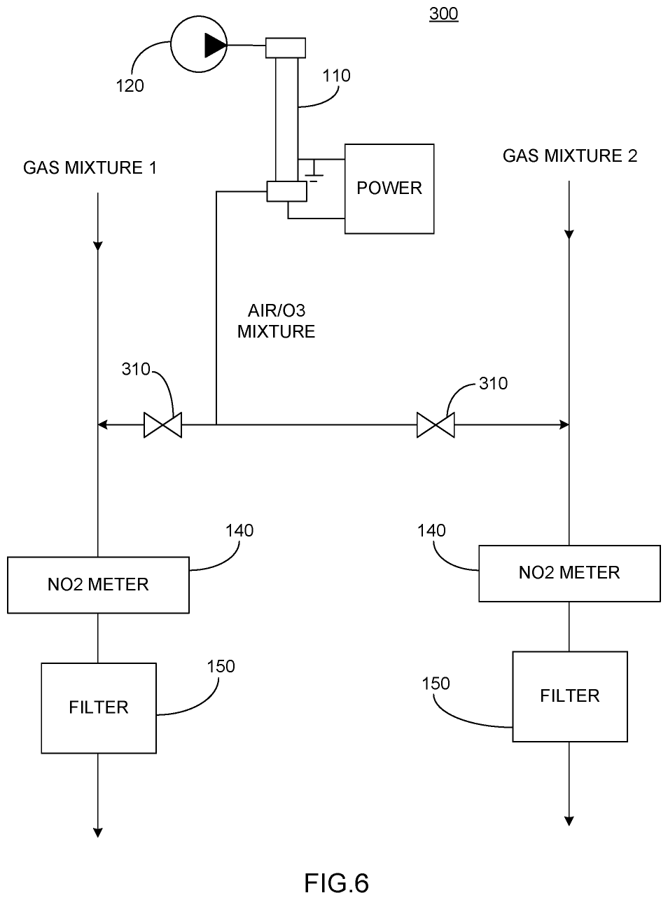

In one embodiment, a single ozone generator 110 can be used for measurements of NO and NO₂ concentration in several independent gas flows as shown in FIG. 6. To do this, ozone flow can be directed by valves with desirable flow rates and be admixed into two or more analyzed flows containing NO and NO₂. The measurement algorithms in this case become equal to those described in the second embodiment. Particularly, FIG. 6 shows a sensor 300 for measuring NO concentration. Sensor 300 comprises: an ozone generator 110; an air pump 120; a pair of NO₂ meters 140; a pair of optional NO₂ and/or NO filters 150; and a pair of valves 310. In one embodiment, each valve 310 comprise As described above in relation to sensor 100, an input of ozone generator 110 is coupled to an output of air pump 120. An output of ozone generator 110 is in fluid communication with an input of each NO₂ meter via a respective valve 310.

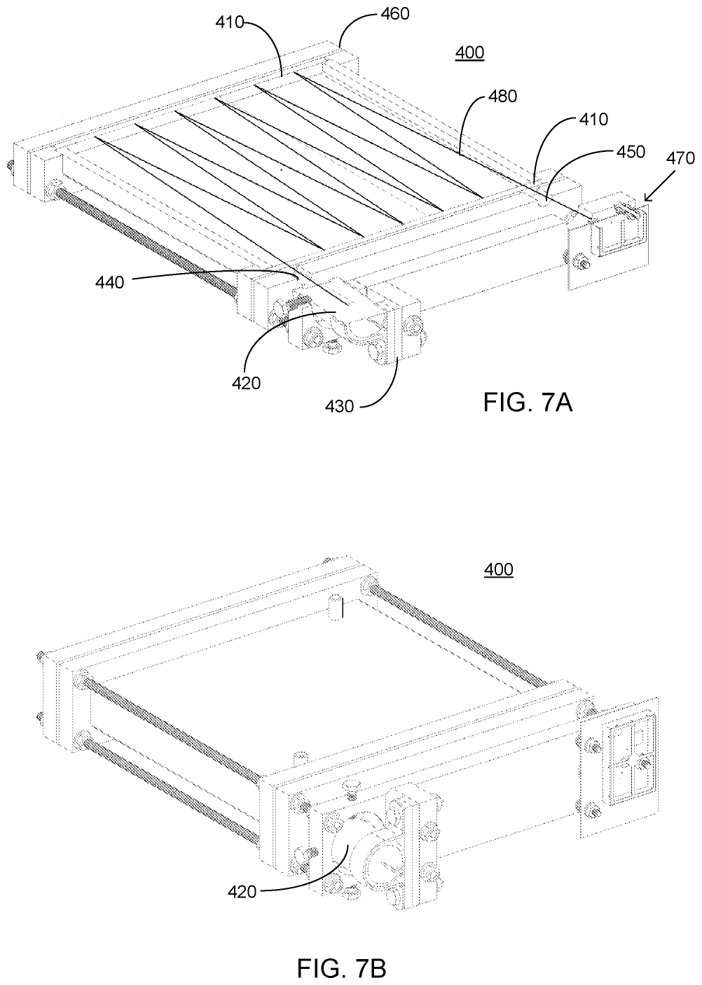

In the embodiment shown in FIGS. 7A-7B, a multi-pass optical cuvette is used. Particularly, FIG. 7A illustrates a cut-away view of a multi-pass optical cuvette system 400 and FIG. 7B illustrates a perspective view of cuvette 400. Cuvette system 400 comprises: a pair of mirrors 410, opposing each other; a light source 420, optionally a laser; a laser adjustment system 430; a beam input channel 440; a beam output channel 450; a sealing 460; and a light sensor 470. The laser beam 480 enters through the beam input channel 440, and is reflected multiple times between mirrors 410, until exiting via beam output channel 450 to be measured by light sensor 470. Such a cuvette is advantageous for measurements of NO₂ and NO concentration in the ppb range instead of ppm. At low concentrations of NO₂, light adsorption is low and requires extra-long optical passes to reach a measurable light intensity drop convenient for reliable measurements. Such low concentrations can be important for reliable measurements of allowable NO₂ concentration in a line which goes to a patient in NO an therapy system or for measurements of exhaled NO concentration in NO diagnostic systems. Parallel mirrors 410 allow a light beam to pass through a sample several times in a small cuvette to reach a desirable optical length which can be more than 10 meters.

Exemplary NO analyzer specifications are described below:

### Example #1

1. Gas analyzer 100 of FIG. 2
2. Measure gas mixture flow rate: 3 l/hour
3. First and second cuvettes optical length: 5 cm
4. LED's wavelength: 400 nm
5. Maximal ozone capacity: 0.2 g/hour
6. Flow rate in ozone infection line: 30l/hour
7. NO₂ measurement range: 10-10000 ppm
8. NO measurement range: 10-10000 ppm

### Example #2

1. Gas analyzer 200 of FIG. 4
2. Measure gas mixture flow rate: 4 l/hour
3. One cuvette with optical length: 5 cm
4. LED's wavelength: 400 nm
5. Maximal ozone capacity: 0.2 g/hour
6. Flow rate in ozone infection line: 4 l/hour
7. NO2 measurement range: 10-10000 ppm
8. NO measurement range: 10-10000 ppm

### Example #3

1. Gas analyzer 300 of FIG. 6
2. Two flows of measured gas mixture with flow rates: 4 l/hour
3. One cuvette with optical length: 5 cm
4. Another multi pass (FIG. 7) cuvette with optical length: 5 m
5. LED's wavelength: 400 nm
6. Maximal ozone capacity: 0.2 g/hour
7. Flow rate in ozone infection line: 4 l/hour
8. First flow:
   NO₂ measurement range: 10-10000 ppm
   NO measurement range: 10-10000 ppm
9. Second flow:
   NO₂ measurement range: 0.1-100 ppm
   NO measurement range: 0.1-100 ppm

As one skilled in the art would recognize as necessary or best-suited for the systems and methods of the disclosure, systems and methods of the disclosure may include computing devices that may include one or more of processor (e.g., a central processing unit (CPU), a graphics processing unit (GPU), etc.), computer-readable storage device (e.g., main memory, static memory, etc.), or combinations thereof which communicate with each other via a bus. Computing devices may include mobile devices (e.g., cell phones), personal computers, and server computers. In various embodiments, computing devices may be configured to communicate with one another via a network.

Computing devices may be used to control the systems described herein including operation of valves and pumps and processing of sensor data from NO sensors, and filter-related sensors.

A processor may include any suitable processor known in the art, such as the processor sold under the trademark XEON E7 by Intel (Santa Clara, CA) or the processor sold under the trademark OPTERON 6200 by AMD (Sunnyvale, CA).

Memory preferably includes at least one tangible, non-transitory medium capable of storing: one or more sets of instructions executable to cause the system to perform functions described herein (e.g., software embodying any methodology or function found herein); data (e.g., data to be encoded in a memory strand); or both. While the computer-readable storage device can in an exemplary embodiment be a single medium, the term “computer-readable storage device” should be taken to include a single medium or multiple media (e.g., a centralized or distributed database, and/or associated caches and servers) that store the instructions or data. The term “computer-readable storage device” shall accordingly be taken to include, without limit, solid-state memories (e.g., subscriber identity module (SIM) card, secure digital card (SD card), micro SD card, or solid-state drive (SSD)), optical and magnetic media, hard drives, disk drives, and any other tangible storage media.

Any suitable services can be used for storage such as, for example, Amazon Web Services, cloud storage, another server, or other computer-readable storage. Cloud storage may refer to a data storage scheme wherein data is stored in logical pools and the physical storage may span across multiple servers and multiple locations. Storage may be owned and managed by a hosting company. Preferably, storage is used to store records as needed to perform and support operations described herein.

Input/output devices according to the disclosure may include one or more of a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, a button, an accelerometer, a microphone, a cellular radio frequency antenna, a network interface device, which can be, for example, a network interface card (MC), Wi-Fi card, or cellular modem, or any combination thereof. Input/output devices may be used to enter desired NO concentration levels and flow rates and to alert users regarding sensor readings and the need for filter replacement.

One of skill in the art will recognize that any suitable development environment or programming language may be employed to allow the operability described herein for various systems and methods of the disclosure. For example, systems and methods herein can be implemented using C++, C #, Java, JavaScript, Visual Basic, Ruby on Rails, Groovy and Grails, or any other suitable tool. For a computing device, it may be preferred to use native xCode or Android Java.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. A sensor for measuring nitric oxide concentration in a sample, the sensor comprising:
an ozone source for oxidizing nitric oxide within a sample to form NO₂; and
one or more light adsorption measurement systems for determining NO₂ levels in the sample in the nitric oxide analyzer before and after oxidizing.

2. The sensor of claim 1, wherein the light adsorption measurement system comprises a light source positioned to pass light through the sample within the sensor.

3. The sensor of claim 2, further comprising a light sensor positioned to receive light from the light source passed through the sample within the sensor.

4. The sensor of claim 2, wherein the light source emits light having a wavelength of about 350 nm to about 400 nm.

5. The sensor of claim 2, wherein the light source comprises one or more LEDs.

6. The sensor of claim 3, further comprising a processor configured to receive adsorption data from the one or more light adsorption measurement systems and determine an NO₂ level therefrom.

7. The sensor of claim 3, wherein the sensor comprises one or more mirrors for reflecting light to pass through the sample one or more times before entering the light sensor, thereby increasing the beam length for measurement of low concentrations of NO₂.

8. The sensor of claim 1, wherein a first light adsorption measurement system is positioned upstream of the ozone source and a second light adsorption measurement system is positioned downstream of the ozone source.

9. The sensor of claim 8, wherein the processor is in communication with the ozone source and is configured to control ozone introduction to the sample through a valve or pump and to determine NO₂ levels before and after introducing ozone to the sample.

10. A method for measuring nitric oxide concentration in a sample, the method comprising:
oxidizing nitric oxide within a volume of sample using ozone to form NO₂;
measuring light adsorption by NO₂ within the sample to determine NO₂ levels in the sample in the nitric oxide analyzer before and after oxidizing; and
subtracting NO₂ levels determined before oxidizing from NO₂ levels determined after oxidizing to determine a nitric oxide concentration in the sample.

11. The method of claim 10, further comprising passing light through the sample from a light source within the sensor.

12. The method of claim 11, further comprising measuring light intensity in light from the light source passed through the sample within the sensor using a light sensor.

13. The method of claim 11, wherein the light source emits light having a wavelength of about 350 nm to about 400 nm.

14. The method of claim 11, wherein the light source comprises one or more LEDs.

15. The method of claim 12, wherein a first light adsorption measurement system is positioned upstream of the ozone source and a second light adsorption measurement system is positioned downstream of the ozone source, the method comprising subtracting NO₂ levels from the first light adsorption measurement system from NO₂ levels from the second light adsorption measurement system.

16. The method of claim 12, further comprising measuring NO₂ levels in the sample, then introducing ozone the sample, then measuring NO₂ levels in the sample again to determine NO₂ levels before and after oxidizing.

17. The method of claim 12, further comprising passing the light through the sample a plurality of times before receiving the light with the light sensor.

18. The method of claim 15, wherein determining nitric oxide levels is according to the formula C2*(C2N/C1N)−C1, where:
C1N is the NO₂ level from the first light adsorption measurement system before introduction of ozone;
C2N is the NO₂ level from the second light adsorption measurement system before introduction of ozone;
C1 is the NO₂ level from the first light adsorption measurement system after oxidation with ozone; and
C2 is the NO₂ level from the second light adsorption measurement system after oxidation with ozone.

19. The method of claim 16, further comprising cyclically introducing ozone to the sample to oxidize the NO₂ therein, wherein determining nitric oxide levels is according to the formula C_{NO}=(Ln(Iₘₐₓ/Iᵢₙ))*Kcal−C_{NO2}, where:
C_{NO}2=(Ln(Iₘₐₓ/Iᵢₙ))*Kcal;
Iᵢₙ is initial light intensity of light passed through the sample at the beginning of an ozone introduction cycle;
Iₘᵢₙ is minimal light intensity of light passed through the sample during the ozone introduction cycle;
Iₘₐₓ is maximal light intensity of light passed through the sample during the ozone introduction cycle; and
Kcal is a calibrating coefficient.
